(19) United States
(12) Patent Application Publication
(10) Pub. No.: US 2022/0378698 A1
(43) Pub. Date: Dec. 1, 2022

(54) PRESERVATIVE FREE PHARMACEUTICAL COMPOSITION FOR OPHTHALMIC ADMINISTRATION CONTAINING CYCLOSPORINE

(71) Applicant: PHARMATHEN S.A., Pallini Attikis (GR)

(72) Inventors: Evangelos KARAVAS, Pallini Attikis (GR); Efthymios KOUTRIS, Pallini Attikis (GR); Vasiliki SAMARA, Pallini Attikis (GR); Ioanna KOUTRI, Pallini Attikis (GR); Anastasia KALASKANI, Pallini Attikis (GR); Andreas KAKOURIS, Pallini Attikis (GR); Rumit Rajivbhai SHAH, Pallini Attikis (GR)

(73) Assignee: PHARMATHEN S.A., Pallini Attikis (GR)

(21) Appl. No.: 17/626,625

(22) PCT Filed: Jul. 12, 2019
(86) PCT No.: PCT/EP2019/025231

§ 371 Date: Jan. 12, 2022

(51) Int. Cl. A61K 9/107; A61K 38/13; A61K 47/44; A61K 47/26

(52) U.S. Cl. CPC A61K 9/107; A61K 38/13; A61K 47/44; A61K 47/26

(57) ABSTRACT

The present invention relates to a stable preservative-free Cyclosporine emulsion in the form of eye drops and a process for the manufacturing thereof, packed in a container that ensures stability of the product for the treatment of keratoconjunctivitis sicca.

### TECHNICAL FIELD OF THE INVENTION

The present invention relates to a preservative free ophthalmic formulation for topical administration containing a therapeutically effective quantity of an immunosuppressive agent such as Cyclosporine to be used for the treatment of keratoconjunctivitis sicca (dry eyes) and the process for its preparation. Such preservative-free formulation is packed in container that ensures physical and chemical stability of the product.

### BACKGROUND OF THE INVENTION

Keratoconjunctivitis sicca (KCS) also known as Dry eye syndrome (DES) is one of the most common problems affecting the general population and can cause problems that range in severity from mildly irritating to debilitating.

Dry eye syndrome is a general term that describes the state of the front of the eye in response to a breakdown in the natural layer of tears that coats the front of the eye, called the tear film. Normally, this layer of tears is a stable, homogenous layer that not only provides the cornea and conjunctiva a healthy buffer from damage were it constantly exposed to the air, but this interface between the tear film and the air is also responsible for a significant amount of the focusing power of the eye. When the tear film becomes unhealthy it breaks down in different places on the cornea and conjunctiva, leading not only to symptoms of irritation, but also to unstable and intermittently changing vision. Other associated symptoms include redness, discharge, and easily fatigued eyes. Blurred vision may also occur. Scarring of the cornea may occur in some cases without treatment.

Inflammation occurring in response to tear film hypertonicity can be suppressed with topical immunosuppressants such as Cyclosporine.

Cyclosporine is an immunosuppressive agent when administered systemically. In patients whose tear production is presumed to be suppressed due to ocular inflammation associated with keratoconjunctivitis sicca, Cyclosporine emulsion is thought to act as a partial immunomodulator. The exact mechanism of action is not known.

The chemical name of Cyclosporine is (3S,6S,9S,12R,15S,18S,21S,24S,30S,33S)-30-Ethyl-33-[(1R,2R,4E)-1-hydroxy-2-methyl-4-hexen-1-yl]-6,9,18,24-tetraisobutyl-3,21-diisopropyl-1,4,7,10,12,15,19,25,28-nonamethyl-1,4,7,10,13,16,19,22,25,28,31-undecaazacyclotritriacontane-2,5,8,11,14,17,20,23,26,29,32-undecone. It is an off-white crystalline solid. Its molecular formula is C₆₂H₁₁₁N₁₁O₁₂ corresponding to a molecular weight of 1202.61. It is slightly soluble in water and saturated hydrocarbons; very soluble in methanol, acetone, and diethyl ether.

EP-B-1142566 discloses topical ophthalmological formulations comprising aqueous solutions containing Cyclosporine, hyaluronic acid or its salt and polysorbate 80.

U.S. Pat. No. 6,555,526 B discloses an ophthalmic pharmaceutical composition comprising trehalose as an effective ingredient and a pharmaceutically-acceptable carrier.

There still remains a need for an effective and safe topical ophthalmic pharmaceutical composition containing Cyclosporine with increased stability, improved solubility and fewer side effects. In particular, there is a need for an ophthalmic composition that is free from preservatives to be provided in a multiple use container and provide efficient dosing of the solution to the patient, without wastage.

### SUMMARY OF THE INVENTION

The main objective of the present invention is to develop a stable, preservative-free ophthalmic formulation comprising Cyclosporine to be used for the treatment of keratoconjunctivitis sicca providing a significant improvement over the prior art formulations.

Moreover, an aspect of the present invention is to provide a preservative free ophthalmic formulation for topical administration containing Cyclosporine which is bioavailable and effective with sufficient self-life.

A further approach of the present invention is to provide ophthalmic solutions that are easily administrable in drop form.

Furthermore, it is an object of the present invention to provide an ophthalmic product that contains no antimicrobial preservatives, it is packed in a multi-dose container that maintains product sterility and is as effective in terms of therapy as products available with preservatives.

It is a further object of the present invention to provide a pharmaceutically effective emulsion suitable for ocular application.

In accordance with the above objects of the present invention, an ophthalmic, preservative-free pharmaceutical emulsion is provided comprising Cyclosporine as active ingredient, a tonicity agent, an emulsifying agent, a viscosity modifying agent, an oily component and one or more pH adjusting agents.

A preferred object of the present invention is to provide a simpler and cost effective process for preparing a stable and sterilized Cyclosporine ophthalmic emulsion.

According to another embodiment of the present invention, a process for the preparation of a preservative-free ophthalmic emulsion containing Cyclosporine is provided and it comprises the following steps:

Preparation of primary emulsion by mixing an oil phase comprising castor oil and Cyclosporine with a water phase comprising polysorbate 80 using high shear or high pressure homogenizer and sterilization by filtration.
Preparation of carbomer copolymer type A phase and sterilization by heat.
Final mixing of primary emulsion and carbomer phase of previous steps using magnetic stirrer or high shear homogenizer.

Other objects and advantages of the present invention will become apparent to those skilled in the art in view of the following detailed description.

### DETAILED DESCRIPTION OF THE INVENTION

For the purpose of the present invention, a pharmaceutical composition comprising an active agent or a combination of active agents is considered “stable” if said agent or combination of agents degrades less or more slowly than it does on its own or in known pharmaceutical compositions.

Ocular administration of drugs is primarily associated with the need to treat ophthalmic diseases. Eye is the most easily accessible site for topical administration of a medication. Ophthalmic preparations are sterile products essentially free from foreign particles, suitably compounded and packaged for instillation into the eye. They are easily administered by the nurse or the patient himself, they have quick absorption and effect, less visual and systemic side effects, increased shelf life and better patient compliance.

Antimicrobial preservatives are added to aqueous preparations that are required to be sterile, such as in ophthalmic solutions. The use of preservatives in topical ophthalmic treatments is ubiquitous for any product that is to be used more than once by the patient as they prevent any microbes that may enter into the product after its first use from allowing those microbes to grow and infect the patient on a later use of the product. Although providing effective biocidal properties with well tolerated short-term use at low concentrations, preservatives can cause serious inflammatory effects on the eye with long-term use in chronic conditions, such as glaucoma or potentially ocular allergies.

Antimicrobial preservatives are not found in single use vials of ophthalmic solutions since they are manufactured aseptically or are sterilized and the products are used once and the dispenser is thrown away.

Preservative-free single dose containers most often are presented as blow-fill-seal (BFS) containers. The user takes the plastic vial and tears or cuts the plastic tip, inverts the vial and squeezes the ophthalmic liquid into the eye. Disadvantages of these systems are linked to the quite complicated filling technology, the need to overfill and amount of material needed for each dose. With an average drop size of ˜35 μl and the standard commercial volume of 400-500 μl, five times the required drug quantity ends up being discarded in case of single dose containers. Additionally, a big amount of packaging material is required associated with high manufacturing costs. A further disadvantage is that, despite numerous technical improvements were made by some manufacturers, the edges around the tip of the opened dropper of disposable, single-dose container are still very sharp, which may cause an accident to the patients eye.

As the use of preservative containing eye drops has been implicated in the development or worsening of ocular surface disease, there is a tendency to limit their use by reducing their concentration as much as possible in eye drops.

Today a range of technical solutions are available to overcome this issue and provide bacterial protection mechanisms. The highest risk of contamination obviously comes from the tip from which the solution exits the container, because it may come in contact with skin and mucosa as well as with infected body fluids. Solutions to prevent contamination via the tip divide into two distinct groups:

1. Containers having “oligodynamic effect” have an open tip release metal ions into the formulation that are toxic to bacteria. Examples include the use of silver wire in the tip of the actuator, a silver coated spring and ball. These components release silver ions into the formulation, which is a time dependent process. The system is able to keep microorganisms down between long dosing intervals, even when the tip is immersed into bacterial contaminated fluid. Silver ions are widely used for their antiseptic properties and even when used for wound dressings, it is safe and no adverse effects are attributed to this treatment. One general limitation of course must be considered: the silver ions may react with certain ions in the formulation and may form precipitates—such as with chloride ions.
2. Containers that use a “mechanical effect” to prevent contamination. Typically this is called “tip seal technology” and is a simple spring loaded valve located directly below the opening of the tip orifice that does not allow any microbes to migrate from any surfaces or contacted liquids into the system; the orifice is sealed under resting conditions. The tip seal keeps the system closed until a defined pressure is reached then the system will open and the formulation is forced through the orifice with a higher pressure than needed to open the valve. When the pressure drops at the end of the actuation the tip seal will immediately close the orifice with an outward movement. So no backflow of potentially contaminated medication or other liquid is possible.

Additionally to protect the integrity of the solution such devices may also have a system to prevent bacteria entering when the system vents. So after use a negative pressure develops inside the container and air may flow back into the container which may carry air born bacteria. Integrity is achieved by a “mechanical effect” and may be one or more of the following:

1. Collapsible internal bag to contain the solution. The use of an internal collapsible bag to contain the systems avoids any negative pressure developing.
2. Filters, these simply filter the air and trap any air born bacteria.
3. Unvented containers—these are containers that do not allow any air to come back into the container at all. Negative pressure continues to build throughout the use of the product without affecting the performance of the container to deliver the solution

The present invention provides ophthalmic formulations that are completely free of preservatives. Such formulations are packed in containers that enable to deliver preservative-free formulations while providing shelf life similar to traditional formulations. The containers of the present invention ensure that medication is kept germ-free even after multiple uses.

Patient compliance is greatly increased as the pumps of the present invention permit them to use preservative-free eye drops without worrying about the potential side effects caused by some preservatives and the related short- and long-term consequences, such as pain or discomfort, foreign body sensation, stinging or burning, dry eye sensation, ocular surface breakdown.

We have found that the design of the tip of the container produce a highly accurate drop size with low variability of drop volume between each drop dispensed.

Therefore, we present as a feature of the present invention a multi-use ophthalmic product comprising a container with an integral bacterial protection system and which has a dispensing tip, wherein the ratio of the inner to the outer diameter of the dispensing tip is from 1:1 to 1:6, and the container having an ophthalmic composition that is dispensed from the tip into the eye of a patient wherein the ophthalmic composition is a preservative-free aqueous solution and contains pharmaceutically acceptable excipients.

Studies of of drop size, assay absorption and actuation force indicated that preservative free packaging of the present invention is suitable to be used with Cyclosporine ophthalmic emulsion.

Emulsion may be defined as a biphasic system consisting of two immiscible liquids usually water and oil, one of which is finely subdivided and uniformly dispersed as droplets throughout the other. Since an emulsion is a thermodynamic system, a suitable emulsifying agent is required to stabilize it. It has two phases: i) oil phase and ii) water phase. In other way i) external phase and ii) internal phase. The phase which makes globules or droplets is known as internal phase or disperse phase and other is external or continuous phase. Oil can be present as internal and external phase and water also as internal or external phase. Emulsion is normally opaque. Particle sizes of emulsion are from 0.1 to 100 μm.

Emulsification is the process by which the dispersed phase is broken up into small droplets. Normally a coarse premix is created by rapid mixing of the ingredients. This is sufficient to break up the dispersed phase into large droplets, and allow adsorption of the emulsifiers prior to final emulsification. According to the present invention two main methods/principles are particularly preferred to homogenize the emulsion. A mechanical method under high shear to break up droplets and high pressure homogenizer that forces the premix through a narrow orifice or valve at high pressures (typically 10-100 MPa). Forcing the emulsion through a valve at high pressure creates turbulence and very high shear forces, thus breaking up the droplets.

Extensive studies have been conducted in order to develop a process that provides a stable and sterilized Cyclosporine emulsion for ophthalmic use that is also economic and feasible for commercial scale preparation. A three-step process was developed comprising the preparation of an emulsion by mixing an oil phase comprising Cyclosporine with a water phase using high shear or high-pressure homogenizer. The preparation of a separate aqueous phase comprising a viscosity enhancing agent and the final mixing of the emulsion and viscosity enhancing agent phase using magnetic stirrer or high shear homogenizer.

The ophthalmic, preservative-free emulsion of the present invention comprises Cyclosporine as active ingredient and one or more other components in amounts adequate to facilitate the effectiveness of the compositions. Examples of such other components include tonicity agents, emulsifying agents, emulsion stabilizing agents, viscosity modifying agents, oily materials that solubilize Cyclosporine, acid and/or bases to adjust the pH of the compositions.

The oily component may be considered the discontinuous phase in the Cyclosporine emulsions of the present invention with the water or aqueous phase being considered the continuous phase in such emulsions. Examples of useful oily materials include olive oil, arachis oil, castor oil, mineral oil. The present invention preferably comprises castor oil in an amount of 1%-2% (w/v), most preferably 1.25% w/v.

In general emulsifier components include a hydrophobic and a hydrophilic component. The emulsifier component must be present in an amount effective in forming the present emulsion and maintaining the oily component in emulsion with aqueous component. Suitable emulsifier components include polysorbate 80, polyalkylene oxide ethers of alkyl alcohols and alkylphenols. The present invention preferably comprises polysorbate 80 in an amount of 0.25%-2% (w/v), most preferably 0.50%-1% w/v.

Useful tonicity agents in the present invention include mannitol, glycerine, sorbitol. The present invention preferably comprises glycerine in an amount of 1.5%-2.5% (w/v), most preferably 2.20% w/v.

The compositions of the present invention include viscosity modifying agents such as cellulose polymers, carbomers, alginates, xanthan gums. Such viscosity modifying agents are employed in an amount effective to provide a desired viscosity to the present compositions. The present invention preferably comprises carbomer copolymer type A that also promotes the stabilization of the emulsion. Carbomer is present in an amount of 0.03%-1% (w/v), most preferably 0.05% w/v.

The pH of the emulsions can be adjusted using sodium hydroxide and/or hydrochloric acid to a physiological pH level. The pH of the emulsions of the present invention is in the range of about 6 to about 10, preferably about 6.5 to about 8 and more preferably from 6.8 to 7.6. The pH can be adjusted in the carbomer phase or in the final product or both.

Pharmaceutical products intended for ophthalmic use must be sterile. Sterilization refers to any process that eliminates, removes, kills, or deactivates all forms of life and other biological agents (such as fungi, bacteria, viruses, spore forms, prions, unicellular eukaryotic organisms such as Plasmodium, etc.) present in a specified region, such as a surface, a volume of fluid, or medication.

Sterilization can be achieved through various means including: heat, chemicals, irradiation, high pressure, and filtration. Sterilization is distinct from disinfection, sanitization, and pasteurization, in that sterilization kills, deactivates, or eliminates all forms of life and other biological agents which are present.

Water at high pressure level is used in moist heat sterilization. Autoclave is the instrument in which this process is carried out. The temperature of the steam in this method is lower when compared with dry heat sterilization, but the high pressure helps with effective sterilization to take place.

Through moist heat sterilization, the most resistant of the spores require a temperature of 121° C. for around half an hour. It is a more effective method when compared with dry heat sterilization. This can be supported by the fact that through moist heat, sterilization can be achieved at lower temperatures in a shorter duration.

In dry heat sterilization, dry heat is used for sterilizing different materials. Heated air or fire is used in this process. As compared to the moist heat sterilization, the temperature in this method is higher. The temperature is usually higher than 356° F. or 180° C. Dry heat helps kill the organisms using the destructive oxidation method. This helps destroy large contaminating bio-molecules such as proteins. The essential cell constituents are destroyed and the organism dies. The temperature is maintained for almost an hour to kill the most difficult of the resistant spores.

Fluids that would be damaged by heat, irradiation or chemical sterilization, such as drug products, can be sterilized by microfiltration using membrane filters. This method is commonly used for heat labile pharmaceuticals. Membrane filters used in production processes are commonly made from materials such as mixed cellulose ester or polyethersulfone (PES). The filtration equipment and the filters themselves may be purchased as pre-sterilized disposable units in sealed packaging or must be sterilized by the user, generally by autoclaving at a temperature that does not damage the fragile filter membranes. To ensure proper functioning of the filter, the membrane filters are integrity tested post-use and sometimes before use. The nondestructive integrity test assures the filter is undamaged and is a regulatory requirement. Typically, terminal pharmaceutical sterile filtration is performed inside of a cleanroom to prevent contamination.

As emulsions are inherently thermodynamically unstable it is very difficult to produce a stable pharmaceutical emulsion that confers adequate treatment to the patient.

The process by which an emulsion completely breaks (coalescence), i.e., the system separates into bulk oil and water phases, is generally considered to be governed by four different droplet loss mechanisms, i.e., Brownian flocculation, creaming, sedimentation flocculation and disproportionation. The first three are the primary methods by which emulsions are destabilized but all four processes may occur simultaneously and in any order.

The present invention has successfully overcome such processing difficulties by applying a step-by-step sterilization in order to obtain a stable and sterile emulsion. More specifically, a primary emulsion was sterilized by filtration and was subsequently mixed with viscosity modifying agent sterilized by heat. The final mixing resulted in a stable emulsion with desired characteristics.

The target of homogenization process is to obtain a primary emulsion with Z-Av of 90-160 nm measured at 1:10 dilution.

The Z-Average size or Z-Average mean used in dynamic light scattering is a parameter also known as the cumulants mean. It is the primary and most stable parameter produced by the technique. The Z-Average mean is the best value to report when used in a quality control setting as it is defined in ISO 13321 and more recently ISO 22412 which defines this mean as the ‘harmonic intensity averaged particle diameter’. The Z-average size will only be comparable with the size measured by other techniques if the sample is monomodal (i.e. only one peak), spherical or near-spherical in shape, monodisperse (i.e. very narrow width of distribution), and the sample is prepared in a suitable dispersant, as the Z-Average mean size can be sensitive to even small changes in the sample, e.g. the presence of a small proportion of aggregates. It should be noted that the Z-average is a hydrodynamic parameter and is therefore only applicable to particles in dispersion or molecules in solution.

### EXAMPLES

Different manufacturing processes were tested in order to obtain stable and sterile product according to table 1 below.

TABLE 1
Composition 1

| Ingredients | % w/v |
|---|---|
| Cyclosporine | 0.050 |
| Castor Oil | 1.250 |
| Polysorbate 80 | 1.0000 |
| Carbomer copolymer type A | 0.050 |
| Glycerin | 2.200 |
| Sodium Hydroxide | q.s to adjust pH to 6.5-8.0 |
| Purified water | q.s to 100 ml |

### A. Terminal Sterilization of Final Product

Composition 1 was manufactured with the following process in an attempt to perform the sterilization at the final product either in an autoclave or with the use of filters.

### Oil Phase

1. Dissolve Cyclosporine in castor oil. Add glycerine in the same phase.

### Aqueous Phase

1. Add polysorbate 80 in 160 ml of water.
2. Add Oil phase drop wise into Aqueous Phase.
Primary Emulsion with High Pressure Homogenizer (HPH)
1. Use the propeller for initial mixing. Stir for 30 min.
2. Use HPH to achieve desired PSD.

### Carbomer Phase

1. Add carbomer copolymer type A in 160 mL of WFI.
2. Adjust pH to 7.00 using NaOH.

### Final Mixing and Terminal Sterilization

1. Mix Primary emulsion with Carbomer Phase and make volume using WFI.
2. Continue stirring to reach target of Z-Avg and Zeta potential.
3. Final product is terminally sterilized at 121° C. for 15 min.

The physicochemical characteristics and chemical stability of the product sterilized with the use of the autoclave are presented in tables 2 & 3 below.

TABLE 2
Physicochemical characterization of
non-sterile and terminally sterilized

| | D10 | D50 | D90 | Z Avg | Zeta | Observa- |
|---|---|---|---|---|---|---|
| Detail | nm | nm | nm | nm | Potential | tion |
| Final product | 148 | 603 | 1660 | 333.7 | −53.5 mv | Stable |
| Non Sterile | | | | | | Emulsion |
| Final product | 10.3 | 20.5 | 170 | 6841 | −13.3 mv | Not stable |
| Autoclave | | | | | | Creaming |
| | | | | | | observed |

TABLE 3
Chemical Stability

| Impurities Results | Results (zero time analysis) |
|---|---|
| Imp. AM (NMT 1.0%) | ND |
| Imp. B (NMT 1.0%) | ND |
| Imp. C (NMT 1.0%) | 0.09% |
| Imp. D (NMT 1.0%) | 0.30% |
| Dihydro Impurity (NMT 1.0%) | 0.32% |
| Any unknown Impurity (NMT1.0%) | 1.60% (RRT 0.24) |
| Total Impurities (NMT 6.0%) | 2.38% |

Results indicate that the final product has higher unknown impurity which are also out of specifications (unknown Impurity at RRT 0.24 above 1.0% according the specifications under Q3B2 ICH Guidance). Also, the globule size distribution (GSD) and Zeta potential of final product were affected and the emulsion system was destabilized (creaming was observed in autoclave sample).

Terminal sterilization of the final product with the use of 0.22 micron filters made out of PES, PVDF and nylon membrane instead of the autoclave in the final step as above was tested as well. The product was not suitable for filtration process. It was observed that filter was blocked immediately. This was attributed to the presence of carbomer copolymer type A that increases the viscosity to final product.

### B. Terminal Sterilization of Primary Emulsion

As terminal sterilization of final product did not give satisfactory results we proceeded with sterilization of primary emulsion. Primary emulsion is made out by mixing the oil phase with the aqueous phase, in the amounts of table 1 presented above.

The primary emulsion was prepared using two different homogenizers (step 1 of “mixing of oil and water phase” see below): High Shear Homogenizer (HSH) and High Pressure Homogenizer (HPH).

The manufacturing process followed for preparing the primary emulsion is described below:

### Oil Phase

1. Dissolve Cyclosporine in castor oil.

### Aqueous Phase

1. Add glycerin and polysorbate 80 in 150 ml of water.
2. Adjust pH to 7.20 using NaOH.

### Mixing of Oil and Water Phase

1. Use the HSH (or HPH) for mixing.
2. Take out 50 ml for rinsing of oil phase beaker.
3. Start stirring of Water phase at high speed and add drop wise Oil Phase into the Water phase.
4. Use 50 ml of rinsing water to take oil droplets.
5. Volume make up to 200 ml.
6. Check and if required adjust pH to 7.20.

### Terminal Sterilization

1. Primary Emulsion is terminally sterilized at 121° C. for 15 min and 121° C. for 8 min to study emulsion stability.

The primary emulsion before heat sterilization was stable regardless the use of HSH or HPH. However, after heat sterilization (either 8 or 15 minutes) the emulsion was not stable, with phase separation and creaming being observed.

### C. Hybrid Process Development

Based on the above findings the manufacturing process was divided into three major steps:

1. Preparation of Primary emulsion and sterilization by filtration.
2. Preparation of Carbomer copolymer type A phase and sterilization by heat.
3. Aseptic Final mixing to achieve stable emulsion with desired characteristics.

### C1. Preparation and Sterilization of Primary Emulsion Using Filtration Process

The primary emulsion was prepared as concentrated phase (table 4) so that at the end, after mixing with Carbomer copolymer type A phase, to prepare final emulsion of Cyclosporine 0.5 mg/ml.

TABLE 4
Composition 2

| Ingredients | % w/v |
|---|---|
| Cyclosporine | 0.25 |
| Castor Oil | 6.25 |
| Polysorbate 80 | 5.00 |
| Glycerin | 11.00 |
| Purified water | q.s to 100 ml |

The process of preparing the primary emulsion is described below

### Oil Phase

1. Dissolve Cyclosporine in castor oil (70° C.). Add glycerin in the same phase.

### Water Phase

1. Add Polysorbate 80 in 120 ml of water (70° C.).
2. Maintain temperature at 70° C. and add oil phase drop wise.
Primary Emulsion with High Shear Mixer (or HPH)
1. Use the HSH (or HPH) for mixing at 70° C.
2. Start stirring of Water phase at high speed and add drop wise Oil phase into the Water phase.
4. Filter Primary emulsion using PES filter.
5. Measure GSD of unfiltered and filtered samples and study emulsion stability.

TABLE 5
Emulsion stability (physical)

| | | | | | |
|---|---|---|---|---|---|
| | Time | D10 | D50 | D90 | Z Avg |
| Sample Details | Interval | nm | nm | nm | nm |

Mixing with HSH

| | | | | | |
|---|---|---|---|---|---|
| Unfiltered Sample | Zero | 155 | 383 | 798 | 319.6 |
| Filtered sample | Time | 148 | 415 | 836 | 324.7 |
| Unfiltered Sample | After | 153 | 386 | 904 | 314.4 |
| Filtered sample | 4 days | 160 | 397 | 804 | 327.6 |
| Unfiltered Sample | After | 156 | 388 | 814 | 317.2 |
| Filtered sample | 7 days | 184 | 390 | 866 | 325.7 |

Mixing with HPH

| | | | | | |
|---|---|---|---|---|---|
| Unfiltered Sample | Zero | 228 | 470 | 1020 | 402.9 |
| Filtered sample | Time | 221 | 469 | 949 | 394.8 |
| Unfiltered Sample | After | 225 | 466 | 919 | 399.4 |
| Filtered sample | 4 days | 236 | 468 | 930 | 396.1 |
| Unfiltered Sample | After | 213 | 478 | 988 | 394.8 |
| Filtered sample | 7 days | 221 | 455 | 1000 | 389.9 |

Results indicate that Primary emulsion prepared using either high shear mixer or high pressure homogenizer is stable as no difference in GSD was observed over period of 7 days in filtered and unfiltered sample. As the primary emulsion is intermediate step of manufacturing process 7 days stability of primary emulsion was studied.

Moreover, Assay analysis (for both HPH and HSH) before and after filtration was performed to check any absorption issue during filtration process. Results (table 6) indicate no significant absorption of Cyclosporine.

TABLE 6
Assay analysis before and after filtration

| | | |
|---|---|---|
| Sample Details | Time Interval | % Assay |

Mixing with HSH

| | | |
|---|---|---|
| Unfiltered Sample | Zero Time | 104.73 |
| Filtered sample | | 101.6 |

Mixing with HPH

| | | |
|---|---|---|
| Unfiltered Sample | Zero Time | 97.24 |
| Filtered sample | | 97.65 |

Additionally, slightly more diluted, in comparison to composition 2, Primary emulsion (Composition 3) was prepared with the same process and studied for GSD and stability of the emulsion.

TABLE 7
Composition 3

| Ingredients | % w/v |
|---|---|
| Cyclosporine | 0.125 |
| Castor Oil | 3.125 |
| Polysorbate 80 | 2.50 |
| Glycerin | 5.50 |
| Purified water | q.s to 100 ml |

TABLE 8
Emulsion stability (physical)

| | | | | | |
|---|---|---|---|---|---|
| | Time | D10 | D50 | D90 | Z Avg |
| Sample Details | Interval | nm | nm | nm | nm |

Mixing with HSH

| | | | | | |
|---|---|---|---|---|---|
| Unfiltered Sample | Zero Time | 67.3 | 184 | 360 | 145.1 |
| Filtered sample | | 65.5 | 186 | 388 | 144 |
| Unfiltered Sample | After 7 days | 74.4 | 184 | 398 | 148 |
| Filtered sample | | 58.4 | 188 | 347 | 142.9 |

Mixing with HPH

| | | | | | |
|---|---|---|---|---|---|
| Unfiltered Sample | Zero Time | 153 | 312 | 595 | 261.7 |
| Filtered sample | | 143 | 312 | 635 | 259.1 |
| Unfiltered Sample | After 7 days | 145 | 304 | 631 | 257.6 |
| Filtered sample | | 148 | 317 | 637 | 261.1 |

Results (table 8) indicate that Primary emulsion prepared using either high shear or high pressure mixing is stable as no difference in GSD was observed over period of 7 days in filtered and unfiltered sample. Moreover, Assay analysis (table 9) before and after filtration was performed to check any absorption issue during filtration process. Results indicate no significant absorption of Cyclosporine.

TABLE 9
Assay analysis before and after filtration

| | | |
|---|---|---|
| Sample Details | Time Interval | % Assay |

Mixing with HSH

| | | |
|---|---|---|
| Unfiltered Sample | Zero Time | 104.32 |
| Filtered sample | | 103.78 |

Mixing with HPH

| | | |
|---|---|---|
| Unfiltered Sample | Zero Time | 104.92 |
| Filtered sample | | 104.09 |

Primary emulsion prepared using High shear or High pressure homogenizer can be filtered and is stable as no difference in GSD is observed in before and after filtration sample.

### C2. Preparation of Carbomer Copolymer Type A Phase and Sterilization by Heat

TABLE 10
Composition 4

| Ingredients | % w/v |
|---|---|
| Carbomer copolymer type A | 0.125 |
| Sodium Hydroxide | q.s to adjust pH to 6.5-8.0 |
| Purified water | q.s to 100 ml |

Composition 4 was prepared according to the following manufacturing process:
1. Add carbomer copolymer type A in 160 mL of WFI (70° C.).
2. Adjust pH to 7.0 using sodium hydroxide.
3. Check pH and viscosity before sterilization.
4. Sterilize carbomer copolymer type A phase by heat.
5. After sterilization check pH and viscosity.

TABLE 11
Chemical parameters before and after sterilization

| Process Step | Viscosity | pH |
|---|---|---|
| Before Sterilization | 250 cps | 7.00 |
| After Sterilization | 200 cps | 7.00 |

Results indicate that no significant difference in viscosity observed for carbomer copolymer Type A phase. Carbomer copolymer Type A phase is stable to sterilization by heat.

C3. Final Mixing to Achieve Stable Emulsion with Desired Characteristics

The products from steps C1 (HSH or HPH) and C2 as above were mixed together in order to obtain the final product (composition 1)

### Final Mixing

1. Mix 120 ml of Primary emulsion with 120 ml of Carbomer Phase at Temp 70° C.
2. Volume make up to 300 ml using WFI.
3. Stir solution using HSH or Magnetic stirrer to achieve desired GSD and Zeta potential.

The Globule Size Distribution of the final product was measured for the final mixing step (mixing using HSH or magnetic stirrer) and in case when the emulsion (C1) is prepared either with HPH or HSH. The measurements were done in various dilutions of the final product in order to see the product's behavior in such case.

TABLE 12
Globe size distribution data

| | | | | |
|---|---|---|---|---|
| | D10 | D50 | D90 | Z Avg |
| Details | nm | nm | nm | nm |

Final Mixing using High shear homogenizer (C1 with HSH)

| | | | | |
|---|---|---|---|---|
| Primary Emulsion Filtered | 148 | 415 | 836 | 324.7 |
| Final product Undiluted | 130 | 456 | 1620 | 282.3 |
| Final product 1:2 Dilution | 72.5 | 220 | 739 | 154.1 |
| Final product 1:4 Dilution | 58 | 163 | 412 | 120 |
| Final product 1:6Dilution | 61.3 | 147 | 384 | 115 |
| Final product 1:8 Dilution | 56.1 | 135 | 293 | 105.6 |
| Final product 1:10 Dilution | 50.9 | 119 | 259 | 100.7 |

Final Mixing using High Shear homogenizer (C1 with HPH)

| | | | | |
|---|---|---|---|---|
| Primary Emulsion Filtered | 126 | 302 | 691 | 250.8 |
| Final product Undiluted | 108 | 524 | 1460 | 288.6 |
| Final product 1:2 Dilution | 80.6 | 222 | 636 | 159.4 |
| Final product 1:4 Dilution | 55 | 189 | 400 | 130.9 |
| Final product 1:6Dilution | 70.2 | 157 | 346 | 122.4 |
| Final product 1:8 Dilution | 57.9 | 148 | 305 | 117.5 |
| Final product 1:10 Dilution | 57.3 | 139 | 326 | 113.1 |

Final Mixing using Magnetic Stirrer (C1 with HPH)

| | | | | |
|---|---|---|---|---|
| Primary Emulsion Filtered | 126 | 302 | 691 | 250.8 |
| Final product Undiluted | 148 | 603 | 1660 | 333.7 |
| Final product 1:2 Dilution | 77.7 | 289 | 664 | 194.2 |
| Final product 1:4 Dilution | 74.2 | 223 | 518 | 160.9 |
| Final product 1:6Dilution | 71.8 | 215 | 446 | 159.2 |
| Final product 1:8 Dilution | 72.1 | 181 | 423 | 141.1 |
| Final product 1:10 Dilution | 61.3 | 148 | 295 | 120 |

Additionally, the physical stability of the product was tested both undiluted and on a 1:10 dilution, for 6 months under accelerated conditions (table 13).

TABLE 13
Final product stability (physical)

| | | | | |
|---|---|---|---|---|
| | Z Avg | D10 | D50 | D90 |
| Tests | nm | nm | nm | nm |

Final Mixing using High shear homogenizer (C1 with HSH)
Undiluted sample

| | | | | |
|---|---|---|---|---|
| Zero Time | 282.3 | 130 | 456 | 1620 |
| 6 Month 40° C. | 249.6 | 114 | 396 | 1450 |

1:10 diluted sample with Water

| | | | | |
|---|---|---|---|---|
| Zero Time | 100.7 | 50.9 | 119 | 259 |
| 6 Month 40° C. | 97.69 | 49.5 | 120 | 262 |

Final Mixing using High Shear homogenizer (C1 with HPH)
Undiluted sample

| | | | | |
|---|---|---|---|---|
| Zero Time | 288.6 | 108 | 524 | 1460 |
| 6 Month 40° C. | 252.8 | 113 | 421 | 1290 |

1:10 diluted sample with Water

| | | | | |
|---|---|---|---|---|
| Zero Time | 113.1 | 57.3 | 139 | 326 |
| 6 Month 40° C. | 104.4 | 51 | 130 | 326 |

Final Mixing using High Shear homogenizer (C1 with HPH)
Undiluted sample

| | | | | |
|---|---|---|---|---|
| Zero Time | 333.7 | 148 | 603 | 1660 |
| 6 Month 40° C. | 379.1 | 140 | 640 | 1630 |

1:10 diluted sample with Water

| | | | | |
|---|---|---|---|---|
| Zero Time | 120 | 61.3 | 148 | 295 |
| 6 Month 40° C. | 130.2 | 71.2 | 158 | 317 |

Additionally, the chemical stability of the product was tested (table 14)

TABLE 14
Final product stability (chemical)

| | | |
|---|---|---|
| Specifications | Zero time | 6 months 40° C |

Final Mixing using High shear homogenizer (C1 with HSH)

| | | | |
|---|---|---|---|
| Assay | 90%-110% | 99.3% | 103.3% |
| Related | Imp. AM NMT 1.0% | Imp. AM ND | Imp. AM ND |
| Substances | Imp. B NMT 1.0% | Imp B 0.09% | Imp. B 0.12% |
| | Imp. C NMT 1.0% | Imp C 0.12% | Imp. C 0.28% |
| | Imp. D NMT 1.0% | Imp D ND Dihydro 0.14% | Imp. D ND Dihydro 0.20% |
| | Dihydro NMT 1.0% | Any unknown | Any unknown |
| | Any unknown NMT 1.0% | RRT0.43 0.11% | RRT0.43 0.33% |
| | Total Impurities NMT 6.0% | Total Impurities 0.49% | Total Impurities 1.09% |

Final Mixing using High Shear homogenizer (C1 with HPH)

| | | | |
|---|---|---|---|
| Assay | 90%-110% | 102.6% | 103.3% |
| Related | Imp. AM NMT1.0% | Imp. AM ND | Imp. AM ND |
| Substances | Imp B NMT1.0% | Imp. B 0.06% | Imp. B 0.09% |
| | Imp. C NMT1.0% | Imp. C 0.16% | Imp. C 0.35% |
| | Imp. D NMT1.0% | Imp. D ND Dihydro 0.15% | Imp. D ND Dihydro 0.17% |
| | Dihydro NMT1.0% | Any unknown | Any unknown |
| | Any unknown NMT 1.0% | RRT0.43 0.10% | RRT0.43 0.35% |
| | Total Impurities NMT6.0% | Total Impurities 0.52% | Total Impurities 1.17% |

Final Mixing using Magnetic Stirrer (C1 with HPH)

| | | | |
|---|---|---|---|
| Assay | 90%-110% | 100.1% | 99.8% |
| Related | Imp. AM NMT1.0% | Imp. AM ND | Imp. AM ND |
| Substances | Imp. B NMT1.0% | Imp. B 0.08% | Imp. B 0.08% |
| | Imp. C NMT1.0% | Imp. C 0.14% | Imp. C 0.17% |
| | Imp. D NMT1.0% | Imp. D ND Dihydro 0.19% | Imp. D ND Dihydro 0.19% |
| | Dihydro NMT1.0% | Any unknown | Any unknown |
| | Any unknown NMT1.0% | RRT 0.43 0.12% | RRT0.43 0.46% |
| | Total Impurities NMT6.0% | Total Impurities 0.61% | Total Impurities 1.23% |

The pH, viscosity, osmolality, specific gravity, surface tension, globule size distribution and zeta potential of compositions prepared according to the present invention were measured using below instrument and methods:

The pH of the compositions was measured according to European pharmacopoeia requirements (Potentiometric determination of pH; 0112005:20203).

The viscosity of the compositions was measured using Rotating viscometer method Brookfield Dv1 Viscometer.

The osmolality of the compositions was measured according to European pharmacopoeia requirements (Osmolality; 01/2005:20235).

The specific gravity of the compositions was measured according to Ph.Eur. 2.2.5.

The surface tension of the compositions was measured via Kross DSAIS easy drop tensiometer.

Globule size distribution and Zeta potential was measured using Malvern Zetasizer (Nano Series) Nano-Zs.

The compatibility of Cyclosporine ophthalmic emulsion in the preservative free packaging was also tested. Final emulsion was filled in preservative free packaging to study the stability of product. The product was found stable in final container.

While the present invention has been described with respect to the particular embodiment, it will be apparent to those skilled in the art that various changes and modifications may be made in the invention without departing from the spirit and the scope thereof, as defined in the claims.

1. A process for the manufacture of an ophthalmic composition of Cyclosporine as the active pharmaceutical ingredient, comprising:
a. preparation of an emulsion by mixing an aqueous phase with an oil phase comprising Cyclosporine and sterilization thereof;
b. preparation of a separate aqueous phase comprising a viscosity modifying agent and sterilization thereof;
c. mixing the emulsion of (a) with the aqueous phase of (b) to obtain the composition and aseptically filling an appropriate ophthalmic container.

2. The process according to claim 1, wherein the mixing of (a) is performed with the use of high shear or high-pressure homogenizer.

3. The process according to claim 1, wherein the sterilization of (a) is performed by filtration.

4. The process according to claim 1, wherein the viscosity modifying agent of (b) is carbomer copolymer type A.

5. The process according to claim 1, wherein the sterilization of (b) is performed by heat.

6. The process according to claim 1, wherein the mixing of (c) is performed with the use high shear homogenizer or magnetic stirrer.

7. The process according to claim 1, wherein the oil phase of (a) comprises castor oil and glycerin.

8. The process according to claim 1, wherein the aqueous phase of (a) comprises polysorbate 80.

9. The process according to claim 1, wherein the pH of the ophthalmic composition of Cyclosporine is adjusted using sodium hydroxide to pH of 6.5 -8.

10. The process according to claim 1, to obtain a preservative-free ophthalmic composition of Cyclosporine.

11. The process according to claim 1, wherein the ophthalmic composition of Cyclosporine is packed in a multi-use container equipped with an integral bacterial protection system.

12. The process according to claim 1, wherein the ophthalmic composition of Cyclosporine possesses globule size distribution with Z-Avg of 90 nm to 160 nm when measured at 1:10 dilution.

13. The process according to claim 1, wherein the ophthalmic composition of Cyclosporine has an average drop volume in the range of 22 μL-39 μL.

14. The process according to claim 1, wherein the ophthalmic composition of Cyclosporine has an average drop volume in the range of 24 μL -34 μL.
